(19) United States
(12) Patent Application Publication
(10) Pub. No.: US 2006/0105405 A1
(43) Pub. Date: May 18, 2006

(54) Migration inhibitory factor in serum as a tumor marker for prostate, bladder, breast, ovarian, kidney, pancreatic and lung cancer and for diagnosis of endometriosis

(75) Inventor: Elliot A. Segal, Bethesda, MD (US)

Correspondence Address: DAVIDSON BERQUIST JACKSON & GOWDEY LLP, 4300 WILSON BLVD., 7TH FLOOR, ARLINGTON, VA, 22203, US

(73) Assignee: Onco Detectors International, LLC, Bethesda, MD (US)

(21) Appl. No.: 11/326,191

(22) Filed: Jan. 6, 2006

Related U.S. Application Data
(63) Continuation-in-part of application No. 11/253,577, filed on Oct. 20, 2005.
(60) Provisional application No. 60/672,015, filed on Apr. 18, 2005.
(60) Provisional application No. 60/619,936, filed on Oct. 20, 2004.
(60) Provisional application No. 60/672,015, filed on Apr. 18, 2005.

(51) Int. Cl. C12Q 1/00

(52) U.S. Cl. 435/7.23; 436/64

(57) ABSTRACT

Macrophage migration inhibitory factor (MIF) has been found to be overexpressed in blood of individuals having various cancers, including prostate, PIN, lung, breast, ovary, kidney, pancreatic and bladder, as well as inflammatory diseases and endometriosis. Normal levels of MIF in serum of males and females without these cancers have been quantified via immunoassay. MIF can be used as a diagnostic tool either by itself or as an adjunct to or in combination with to conventional diagnostic tests.

### CROSS-REFERENCE TO RELATED APPLICATIONS

This application claims the benefit of U.S. Provisional Application Ser. No. 60/672,015, filed Apr. 18, 2005 and is a continuation-in-part of U.S. patent application Ser. No. 11/253,577, filed Oct. 20, 2005, which claims the benefit of U.S. Provisional Application Ser. No. 60/619,936, filed Oct. 20, 2004 and U.S. Provisional Application Ser. No. 60/672,015, filed Apr. 18, 2005. The entire contents of each of these applications are herein incorporated by reference.

### FIELD OF THE INVENTION

The invention relates to the diagnosis of prostate, bladder, breast, lung, ovarian, pancreatic and kidney cancer, and to the diagnosis of endometriosis, by measuring migration inhibitory factor (MIF) in blood, including serum and plasma as a diagnostic test, either alone or as an adjunct to or in conjunction with other diagnostic tests such as prostate specific antigen (PSA) and TNF-α (or TNF-alpha).

### SUMMARY AND DETAILED DESCRIPTION OF THE INVENTION

The proinflammatory cytokine, macrophage migration inhibitory factor (MIF) has been associated with cancer angiogenesis, progression and metastasis in a number of adenocarcinomas, including prostate cancer (CaP), and recently has been found to be secreted by human bladder cancer cells. For example, elevated serum levels of MIF has been found in men with CaP and prostatic intraepithelial neoplasia (PIN). In this regard, reference is made to: (i) Provisional patent application No. 60/547,052 (“Methods for Diagnosing and Treating Bladder Cancer”, VA ID 03-161); (ii) Provisional patent application No. 60/532,889 (“Macrophage Migration Inhibitory Factor (MIF) as a Marker for Urological Inflammatory Disease”, VA ID 03-079); (iii) U.S. patent application Ser. No. 10/644,797 and PCT/US04/05288 (“Serum Macrophage Migration Inhibitory Factor (MIF) as a Marker for Prostate Cancer”, VA ID 03-44).

The advantage of using MIF as a diagnostic tool is that its presence can be detected and measured in the blood, making it a relatively non-invasive diagnostic procedure.

I utilized an improved modified ELISA test that utilizes a 1:10 dilution where serum MIF exhibits higher sensitivity as a marker for prostate cancer than PSA and therefore can be used either as a surrogate for PSA or used as an adjunct or in conjunction with PSA to diagnose prostate cancer. Although I found that a 1:10 dilution was the most preferred in the ELISA immunoassay, I found that other dilutions could be utilized as well, such as 1:1 to 1:40. Other diagnostic tests including TNF-α can also be utilized as an adjunct or in conjunction with MIF and/or PSA for improved non-invasive diagnostic detection of prostate cancer. Current results also show that MIF levels measured in blood, for example serum and plasma, MIF can be used as a diagnostic tool to detect endometriosis as well as cancer, including bladder, lung, pancreatic and kidney cancer in men and women, and breast cancer and ovarian cancer in women. It is believed that this is the first time that elevated MIF levels in blood, specifically serum, has been recognized as a diagnostic tool for bladder, lung, kidney, breast, pancreatic and ovarian cancers, and for endometriosis. In addition, MIF levels were measured in Plasma-EDTA, citrate, and heparin in control patients and therefore plasma could be used as a substitute for serum in detecting prostate, bladder, lung, kidney, breast, pancreatic and ovarian cancer.

Indeed, in addition to the above, this invention will also constitute a novel and improved, relatively non-invasive, confirmatory test for the presence of cancer, especially where other indicators of specific cancers may be present or otherwise determinable.

Archived serum samples which had been previously analyzed for PSA levels from biopsy-confirmed CaP, PIN and normal men were obtained. These archival serum samples were then tested for MIF levels using the specifically modified ELISA (Enzyme Linked Immunosorbent Assay) immunoassay. It should be noted that immunoassays other than ELISA could also be used to test for MIF levels in serum, plasma and urine. These alternative immunoassays include those such as Point of Care or Rapid Assays; Lateral Flow Assays, Multiplex Analyzers for Immunoassay; Solid-Phase and Liquid Phase Assays, among others. Automated analyzers could also be used to measure MIF levels in accordance with this invention.

In these studies, frozen archived serum was used. Fresh serum could also be utilized.

Archived serum samples were also obtained from individuals known to have lung, ovarian, kidney, breasts pancreatic or bladder cancer and MIF levels were measured and compared with archived serum samples from individuals believed to be cancer-free (the Control Group). In all these instances, a statistically significant overexpression of MIF was observed in the serum from the individuals having cancer as compared to the control group, leading to a conclusion that serum MIF is a useful diagnostic marker for these types of cancer either used alone or in conjunction with other tests. Similar testing using modified Elisa methodology has also proven useful in diagnosing and/or screening for endometriosis.

Materials and Protocol Utilized in Evaluation of Serum Samples for MIF

A modified ELISA immunoassay methodology was utilized in these tests. Unlike prior ELISA testing, a specific one to ten dilution was found to be optimal and therefore utilized, but other dilutions can be utilized including, for example, 1:1 to 1:40 dilutions. However, I found that the specific one to ten dilution increased the accuracy of the readings and thereby yielded significantly better comparison results between the specific cancer groups and the control groups.

The following materials were prepared: (1) buffer; (2) MIF Standard; (3) Goat anti-human MIF-Biotin Conjugate Reagent; and (4) Streptavidin-HRP Conjugate Reagent.

To make the buffer, 0.5% (w/v) solution of bovine serum albumin (BSA)/Phosphate buffered saline (PBS) was prepared. 50 ml of 1.0% (w/v) BSA/PBS was mixed with 50 ml PBS to yield 100 ml of 0.5% (w/v) BSA/PBS.

The human MIF lyophilized standard stock was reconstituted with 0.2 ml of 1% BSA in PBS to yield 10,000 ng/ml. The standard was then mixed to ensure complete reconstitution and allowed to sit for 20 minutes at 4° C. A 10× dilution was then made from the 1,000 ng/ml solution to yield 1,000 ng/ml MIF standard solution by mixing 0.005 ml of 10,000 ng/ml stock with 0.045 ml of 0.75% ProClin 300 (from Supelco) with 0.5% (w/v) BSA/PBS to yield 0.05 ml. The solution was mixed well. Then 10 ng/ml MIF solution was made from the 10,000 ng/ml stock by mixing 0.01 ml of 1,000 ng/ml MIF with 0.990 ml of diluent to yield 1.0 ml which was mixed well. A serial dilution was then prepared of 5 ng/ml to 0.156 ng/ml using 0.5 ml from the previous dilution plus 0.5 ml of appropriate diluent to yield 1.0 ml. The resulting solution was mixed well.

Goat anti-human MIF-Biotin Conjugate Reagent was prepared by spiking 0.1 mg/ml of normal mouse IgG and 0.1 mg/ml normal IgG in 1% BSA/PBS buffer. 0.60 ml of Normal mouse IgG (10.0 mg/ml) and 0.56 ml of Normal Goat IgG (10.72 mg/ml) was added to 58.85 ml of 1% BSA/PBS to yield 60.0 ml. The solution was then mixed well. An amount of anti-human MIF-Biotin Conjugate (1:800 antibody stock at 36 ng/ml or 1:1,100 antibody stock at 50 ng/ml) in BSA/PBS buffer was prepared by adding 0.0165 ml of anti-human MIF-Biotin to 13.18 ml of 1% BSA/PBS and mixing well to yield a mix of 45 ng/ml after dilution.

The Streptavidin-HRP Conjugate Reagent was prepared by preparing a 1:800 Streptavidin-HRP in 1% BSA/PBS by mixing 0.0165 ml Streptavidin-HRP with 13.18 ml of 1% BSA/PBS yielding 13.2 ml. The solution was mixed well and stored under dark conditions until it is used.

After the materials were prepared, assays were run using archived frozen serum from the control groups and from those who have the clinically diagnosed cancer of interest. For the purposes of this study, males and females in the control groups were tested separately and consisted of individuals who were apparently cancer-free. One hundred forty one (141) control males were tested; one hundred eighty four (184) control females were tested.

Standard curves were prepared in duplicate followed by 1:10 pre-diluted normal female or normal male serum samples in singlet.

Additional plates utilized a standard curve in duplicate followed by 1:10 pre-diluted cancer serum samples (from patients with clinically diagnosed prostate, breast, lung, kidney, breast or ovarian cancer) in singlet. The desired number of coated wells in the microtiter plate were secured. 100 micro liter of MIF Standard and the pre-diluted sample were added in each well and then the walls were covered with adhesive strip. The microtiter plates were incubated for two hours at room temperature (18° C.-25° C.) on an orbital shaker set at a speed of approximately 750 rpm. The wells were then washed and rinsed four times with the 1× washing buffer and one time with deionized water. The microtiter plate was placed on absorbent paper towels to remove any residual water from the wash. 100 micro liters of MIF detection antibody was added to each well. The microtiter plate was covered with a new adhesive strip and incubated for two hours at room temperature on the shaker at a speed of approximately 750 rpm. Thereafter, the wells were washed and rinsed four times with the 1× washing buffer and one time with deionized water. The microtiter plate was placed on absorbent paper towels to remove any residual water from the wash. 100 microliter of Streptavid-HRP was added to each well. The microtiter plate was incubated for 20 minutes at room temperature on an orbital shaker with a speed set at approximately 750 rpm. The wells were washed and rinsed four times with the 1× washing buffer and one time with deionized water. The microtiter plate was placed on absorbent paper towels to remove any residual water from the wash. 100 microliter TMB Reagent (BioFX) was dispensed into the wells. The microtiter plate was incubated for 20 minutes at room temperature with mechanical shaking at a speed of approximately 750 rpm. 100 microliters of 1 N HCl was dispensed into the wells. The microtiter plate was agitated for twenty seconds. The absorbance was read at 450 nm within 20 minutes.

The manufacturing of the assay and testing for serum levels of MIF in known cancer patients and controls were conducted under FDA GMP practices in a GMP facility.

The foregoing represents a typical preparation. However, it should be understood that there can be several variations of the above, including in the ranges of the amount or amounts of the materials utilized, in the incubation times and in component variations.

Testing Control Subjects

A total of 141 males with no apparent cancer (Male Control Group) were tested using the modified ELISA protocol described above. Archived frozen serum was used for analysis. The mean MIF was 4.7 ng/ml; the median MIF was 2.2 ng/ml.

A total of 184 females with no apparent cancer (Female Control Group) were tested using the modified ELISA protocol described above. Archived frozen serum was used for analysis. The mean MIF was 5.7 ng/ml; the median MIF was 1.9 ng/ml.

MIF Testing for Prostate Cancer and PIN

Archived serum samples which had been previously analyzed for PSA levels from biopsy confirmed male samples were obtained. Ninety-two patients with biopsy-confirmed CaP were tested for serum MIF and also tested using the UltraSensitive PSA kit. While the standard threshold for MIF levels had not previously been determined, I have now determined a novel and appropriate MIF threshold based upon statistical analysis of data obtained from both cancer patients and the Control group. These results show that a value of about 2.6 ng/ml is an appropriate MIF threshold. The results of the study are set forth in the following Tables 1, 2, 3 and 4.

TABLE 1
MIF Positives as Predictor of Prostate Cancer

| MIF | Prostate Ca | Normal | Totals |
|---|---|---|---|
| >2.6 ng/ml | 82 | 6 | 88 |
| <2.6 ng/ml | 10 | 16 | 26 |
| Totals | 92 | 22 | |

SENSITIVITY = 89.1
SPECIFICITY = 72.3
POSITIVE PREDICTIVE VALUE = 93.2%
NEGATIVE PREDICTIVE VALUE = 61.5%
Note:
There were 92 Prostate Cancer patient samples available for the MIF analysis, and 91 samples were available for the PSA and TNF-α analyses. Combination tables utilized 91 patient samples.

TABLE 2
PSA Positives as Predictor of PSA

| | Clinical Status | | |
|---|---|---|---|
| PSA | Prostate Ca | Normal | Totals |
| >4.0 ng/ml | 44 | 2 | 46 |
| <4.0 ng/ml | 47 | 20 | 67 |
| Totals | 91 | 22 | |

SENSITIVITY = 48.3%
SPECIFICITY = 90.1%
POSITIVE PREDICTIVE VALUE = 95.7%
NEGATIVE PREDICTIVE VALUE = 29.9%

TABLE 3
Dual MIF and PSA Positives as Predictor of Prostate Cancer

| MIF and | Clinical Status | | |
|---|---|---|---|
| PSA | Prostate Ca | Normal | Totals |
| Pos | 41 | 2 | 43 |
| Neg | 50 | 20 | 70 |
| Totals | 91 | 22 | |

SENSITIVITY = 45.1%
SPECIFICITY = 90.9%
POSITIVE PREDICTIVE VALUE = 95.3%
NEGATIVE PREDICTIVE VALUE = 28.6%

TABLE 4
Total MIF and PSA Positives as Predictor of Prostate Cancer

| MIF | | | |
|---|---|---|---|
| and/or | Clinical Status | | |
| PSA | Prostate Ca | Normal | Totals |
| Pos | 86 | 7 | 93 |
| Neg | 6 | 15 | 21 |
| Totals | 92 | 22 | |

SENSITIVITY = 93.8%
SPECIFICITY = 68.2%
POSITIVE PREDICTIVE VALUE = 92.5%
NEGATIVE PREDICTIVE VALUE = 71.4%

The results, as set forth in Tables 1, 2, 3, and 4, indicate that MIF and PSA are independent biomarkers for the prediction of prostate cancer. MIF has a higher sensitivity for the detection of prostate cancer than PSA. However, MIF has a lower specificity, presumably because of its prevalence in other disease states (such as inflammatory diseases and endometriosis) and in other cancers. In addition, the data shows that dual PSA/MIF positives do not increase the sensitivity of either when tested alone; however when both tests are positive, the specificity is higher than one or the other. Finally, the use of both biomarkers increases the sensitivity for the prediction of prostate cancer. At a minimum, MIF augments the use of PSA. Using MIF as a marker using a threshold of 2.6 ng/ml, significantly reduces false results (both false negatives and false positives) of PSA, when the PSA threshold is set at 4.0 ng/ml which is the current accepted clinical standard.

TNF-α was also tested separately as yet a third biomarker that could further serve as an adjunct for a panel of tests including PSA and MIF. The TNF-α test utilized was the modified ELISA immunoassay. Samples with TNF-α readings greater than about 10 pg/ml were considered to be positive indicators for prostate cancer.

Twenty two normal (control) samples from males and 91 biopsy confirmed prostate cancer patients samples were examined using MIF, PSA and TNF-α. The threshold, or cut off point for PSA was 4 ng/ml; for MIF it was about 2.6 ng/ml and for TNF-α it was about 10 pg/ml. It should be noted that clinicians generally utilize normal levels for PSA at 4 ng/ml, although some clinicians choose a lower level such as 2.4 ng/ml. Based on the data obtained from the 141 individuals tested, with MIF set at about 2.6 ng/ml, the mean was 4.7 ng/ml the median was 2.2 ng/ml and the standard deviation was 6.6. Results of that study are set forth in Tables 5-9.

Finally, a comparison of serum samples from 91 patients positive for prostate cancer were compared with serum samples from 22 controls using TNF-α, MIF and PSA. Results are set forth in Table 10.

I reviewed the Male Control and the Prostate Confirmed clinical samples in seven categories: (1) PSA only; (2) MIF only; (3) TNF-α only; (4) PSA with MIF; (5) PSA with TNF-α; (6) MIF with TNF-α; (7) PSA with MIF and TNF-α. In analyzing data from the Male Control Group, if the PSA level was below 4 ng/ml, it was deemed correct since the finding was consistent with the clinical determinations; if above 4 ng/ml, it was deemed incorrect (false positive). In the Male Control Group, if the MIF level was below about 2.6 ng/ml, it was deemed correct; if it was above about 2.6 ng/ml, it was deemed incorrect (false positive). In the Male Control Group, if the TNF-α was below about 10 pg/ml, it was deemed correct; if it was above 10 pg/ml, it was deemed incorrect (false positive). In the Prostate Confirmed Group, if the PSA level was above 4 ng/ml it was deemed correct; if below 4.0 ng/ml deemed incorrect (false negative). In the Prostate Confirmed Group, if the MIF level was above about 2.6 ng/ml it was deemed correct; if below about 2.6 ng/ml deemed incorrect (false negative). In the Prostate Confirmed Group, if the TNF-α level was above about 10 pg/ml it was deemed correct; if below about 10 pg/ml deemed incorrect (false negative). Tables 5-10 show the results from these tests:

TABLE 5
TNF-α Positives as Predictor of Prostate Cancer

| | Clinical Status | | |
|---|---|---|---|
| TNF-α | Prostate Ca | Normal | Totals |
| Pos | 35 | 4 | 39 |
| Neg | 56 | 18 | 74 |
| Totals | 91 | 22 | |

SENSITIVITY = 38.4%
SPECIFICITY = 81.8%
POSITIVE PREDICTIVE VALUE = 89.9%
NEGATIVE PREDICTIVE VALUE = 24.0%

TABLE 6
Dual TNF-α and PSA Positives as Predictor of Prostate Cancer

| TNF-α | Clinical Status | | |
|---|---|---|---|
| and PSA | Prostate Ca | Normal | Totals |
| Pos | 16 | 0 | 16 |
| Neg | 75 | 22 | 97 |
| Totals | 91 | 22 | |

SENSITIVITY = 17.8%
SPECIFICITY = 100%
POSITIVE PREDICTIVE VALUE = 100%
NEGATIVE PREDICTIVE VALUE = 22.7%

TABLE 7
Dual TNF-α and MIF Positives as Predictor of Prostate Cancer

| TNF-α | Clinical Status | | |
|---|---|---|---|
| and MIF | Prostate Ca | Normal | Totals |
| Pos | 31 | 2 | 33 |
| Neg | 60 | 20 | 80 |
| Totals | 91 | 22 | |

SENSITIVITY = 34.0%
SPECIFICITY = 90.1%
POSITIVE PREDICTIVE VALUE = 93.4%
NEGATIVE PREDICTIVE VALUE = 25.0%

TABLE 8
Total TNF-α and PSA Positives as Predictors of Prostate Cancer

| TNF-α | | | |
|---|---|---|---|
| and/or | Clinical Status | | |
| MIF | Prostate Ca | Normal | Totals |
| Pos | 63 | 0 | 63 |
| Neg | 28 | 22 | 50 |
| Totals | 91 | 22 | |

SENSITIVITY = 69.2%
SPECIFICITY = 100%
POSITIVE PREDICTIVE VALUE = 100%
NEGATIVE PREDICTIVE VALUE = 44.%

TABLE 9
Total TNF-α and MIF Positives as Predictors of Prostate Cancer

| TNF-α | | | |
|---|---|---|---|
| and/or | Clinical Status | | |
| MIF | Prostate Ca | Normal | Totals |
| Pos | 84 | 8 | 92 |
| Neg | 7 | 14 | 21 |
| Totals | 91 | 22 | |

SE
SENSITIVITY = 92.3%
SPECIFICITY = 63.7%
POSITIVE PREDICTIVE VALUE = 91.3%
NEGATIVE PREDICTIVE VALUE = 66.7%

TABLE 10
Total TNF-α, MIF and PSA Positives as Predictors of Prostate Cancer

| TNF-α, | | | |
|---|---|---|---|
| and/or MIF | Clinical Status | | |
| and/or PSA | Prostate Ca | Normal | Totals |
| Pos | 87 | 0 | 87 |
| Neg | 4 | 22 | 26 |
| Totals | 91 | 22 | |

SENSITIVITY = 95.6%
SPECIFICITY = 100%
POSITIVE PREDICTIVE VALUE = 100%
NEGATIVE PREDICTIVE VALUE = 84.6%

In this study, PSA had two false positives; MIF identified one of those false positives as negative, and TNF-α identified both of those false positives as negative. However, MIF and TNF-α each identified four false positives.

This study shows that any combination of two biomarkers provides an improved measure of differentiating between normal and prostate cancer patients over the current standard of testing using PSA alone. The combination of three biomarkers (PSA, and MIF and TNF-α) provides the most improved measure (96% sensitivity and 100% specificity) of differentiating between normal and prostate cancer patients over the current standard of testing using PSA alone.

The results of this study and the data set forth in Tables 1-10 lead to the conclusions that MIF, PSA and TNF-α are independent biomarkers for the prediction of prostate cancer, and collectively are a better and more predictive set of biomarkers. Of the three tests, it can be seen that MIF has higher sensitivity for the detection of prostate cancer than PSA, but less specificity because of its prevalence in other disease states. Of the three tests, TNF-α has the lowest sensitivity of the three markers when used alone.

Dual testing increases overall specificity. Specifically, dual PSA/MIF positives do not increase the sensitivity of either when tested alone, however when both are positive, the specificity is high. In addition, dual MIF/TNF-α positives do not increase the sensitivity of either when tested alone, however when both are positive the specificity is high. Finally, dual PSA/TNF-α positives do not increase the sensitivity of either when tested alone, however when both are positive the specificity is high. The following conclusions on dual testing were reached with respect to sensitivity and specificity. First, when either MIF or PSA is positive in the same sample the sensitivity is high and the specificity is relatively high. Second, when either MIF or TNF-α is positive in the same sample the sensitivity is high and the specificity is relatively high. Third, when either PSA or TNF-α is positive in the same sample the sensitivity is moderate and the specificity is extremely high. Fourth, when one or more of MIF or TNF-α or PSA is positive in the same sample the sensitivity and specificity for prostate cancer detection is at its highest.

TABLE 11

| | Sensitivity | Specificity | PPV | NPV |
|---|---|---|---|---|
| PSA Alone | 48 | 90 | 96 | 30 |
| MIF Alone | 89 | 72 | 93 | 61 |
| TNF-α Alone | 38 | 82 | 90 | 24 |
| PSA & MIF | 45 | 91 | 95 | 28 |
| TNF-α & MIF | 34 | 90 | 93 | 25 |
| TNF-α & PSA | 17 | 100 | 100 | 23 |
| PSA and/or MIF | 94 | 68 | 93 | 71 |
| TNF-α and/or MIF | 92 | 63 | 91 | 67 |
| TNF-α and/or PSA | 69 | 100 | 100 | 44 |
| PSA and/or MIF and/or TNF-α | 96 | 100 | 100 | 85 |

The current “Gold Standard” serum assay for prostate cancer is PSA. In my studies PSA alone had a sensitivity of 48% and a specificity of 90%. Utilizing the 3 panel assay adding in MIF and TNF-α we found superior results in several panel configurations.

The PSA sensitivity of 48% was exceeded significantly as follows:

| | |
|---|---|
| MIF alone = | 89% |
| PSA and or MIF = | 94% |
| TNF-α and or MIF = | 92% |
| PSA and/or MIF and/or TNF-α = | 96% |

The best predictor of CaP is therefore the three panel test.

The PSA specificity of 90% was exceeded as follows:

| | |
|---|---|
| PSA and MIF = | 91% |
| TNF-α and PSA = | 100% |
| TNF-α and or PSA = | 100% |
| PSA and/or MIF and or TNF-α = | 100% |

Other Diagnostic Findings for Cancer Diagnosis of Blood and Urine with MIF
MIF Testing for PIN

In addition, archived serum samples from ten males having clinically diagnosed prostatic intraepithelial neoplasia (PIN) were found to have MIF overexpressed compared to the Male Control Group. Ten PIN serum samples were tested for MIF and found to have a mean of 9.5 ng/ml as compared with 4.7 ng/ml for the Male Control Group. The PIN samples had a median MIF of 6.5 ng/ml as compared with 2.2 ng/ml for the Male Control Group. This leads to the conclusion that serum MIF is an effective biomarker for the diagnosis of PIN.

MIF Testing for Bladder Cancer Using Serum

Studies on 74 males having clinically diagnosed bladder cancer revealed that MIF is statistically overexpressed in serum. Although previous studies (U.S. Provisional Patent application No. 60/547,052) recognized that MIF is overexpressed in urine in bladder cancer, this is the first study to show overexpression of MIF in serum associated with bladder cancer.

Archived serum from the 74 males with bladder cancer was tested using the ELISA methodology described above and compared with the Male Control Group. The cancer group had a mean serum MIF of 13.3 ng/ml compared with a mean of 4.7 ng/ml in the Male Control Group. The cancer group had a median serum MIF of 10.2 ng/ml, which was statistically significantly higher than the 2.2 ng/ml median level of the Male Control Group.

Similar studies were also conducted on 21 females having clinically diagnosed bladder cancer using the ELISA methodology described above. The cancer group had a mean serum MIF level of 14.6 compared with a mean of 5.7 ng/ml in the Female Control Group. The females with diagnosed bladder cancer had a median level of 8.4 ng/ml, which was statistically significantly higher than the median level of 1.9 ng/ml for the Female Control Group.

Thus, MIF in serum is an effective biomarker to predict bladder cancer used either alone or as an adjunct screening assay with urine cytology, which is currently the most commonly used noninvasive screening tool. Conventional urine cytology sensitivity is quite low, in the vicinity of 20%.

MIF Testing for Breast Cancer

Eighty two females with clinically diagnosed breast cancer were tested for MIF levels. Using the ELISA methodology described above, the cancer group was found to have a mean serum MIF level of 6.4 ng/ml compared with a mean of 5.7 ng/ml for the Female Control Group. The females with diagnosed breast cancer had a median MIF level of 3.6 ng/ml, as compared to the median level of 1.9 ng/ml for the Female Control Group. MIF can therefore be used to screen for breast cancer, either by itself or as an adjunct to screening mammography.

MIF Testing for Ovarian Cancer

Six females with clinically diagnosed ovarian cancer were tested for MIF levels using archived frozen serum samples. Using the ELISA methodology described above, the cancer group was found to have a mean serum MIF level of 4.3 ng/ml compared with a mean of 5.7 ng/ml for the Female Control Group. The females with ovarian cancer had a median level of 3.3, ng/ml, as compared to the median level of 1.9 ng/ml for the Female Control Group.

MIF Testing for Kidney Cancer

Twenty one archived frozen serum samples from individuals with clinically diagnosed kidney cancer were analyzed. The gender of the individuals was unknown. The mean level was 11.9 ng/ml compared with 4.7 ng/ml for the Male Control Group and 5.7 ng/ml for the Female Control Group. The median level for the cancer group was 8.7, compared with 2.2 for the Male Control Group and 1.9 for the Female Control Group.

MIF Testing for Lung Cancer

Eighteen archived frozen serum samples from individuals with clinically diagnosed lung cancer were analyzed. There were 8 Males and 10 Females. The mean level for Males with lung cancer was 7.9 ng/ml compared with 4.7 ng/ml for the Male Control Group. The mean level for Females with lung cancer was 6.9 ng/ml compared with 5.7 ng/ml for the Female Control Group. The median level for the cancer group was 5.2 ng/ml for cancer Males compared with 2.2 for the Male Control Group and 4.5 ng/ml for cancer Females compared to 1.9 for the Female Control Group.

MIF Testing for Cancer Using Plasma

Studies on 27 normal male patients and 33 normal female patients utilizing the ELISA immunoassay employing a 1:10 dilution revealed that MIF was present in Plasma-EDTA, Citrate and Heparin. Thus MIF can be measured in blood and specifically in several varying media of plasma. Finding levels of MIF in control patients in comparable levels to MIF levels of control patients in serum suggests MIF can be a useful biomarker in plasma as well as serum.

MIF Testing for Pancreatic Cancer and Endometriosis

Pancreatic Cancer

Seven archived frozen serum samples from individuals with clinically diagnosed pancreatic cancer were analyzed for MIF levels by utilizing the modified ELISA immunoassay employing a 1:10 dilution. The gender of the individuals was unknown. The mean MIF level of the cancer group was 4.8 ng/ml compared with an MIF level of 4.7 ng/ml for the Male Control Group and of 5.7 ng/ml for the Female Control Group. The median MIF level for the cancer group was 3.6, compared with 2.2 for the Male Control Group and 1.9 for the Female Control Group. One of the MIF levels among the seven cancer samples was below 2.6 ng/ml, the cut point for cancer. Hence, MIF levels for 86% of the cancers were correctly identified. Given the small sample size, the finding of greater levels of MIF in the median sample of those patients with pancreatic cancer compared to the median level of MIF of the control group suggests MIF can be a useful biomarker for pancreatic cancer.

Endometriosis

Twenty females with clinically diagnosed endometriosis were tested for MIF levels. Using the modified ELISA methodology described above, the diagnosed group was found to have a mean serum MIF level of 4.7 ng/ml compared with a mean MIF level of 5.7 ng/ml for the Female Control Group. The females with diagnosed endometriosis had a median MIF level of 3.4 ng/ml, as compared to the median level of 1.9 ng/ml for the Female Control Group. MIF levels for sixteen of the twenty endometriosis cases were above 2.6 ng/ml, the cut point for identifying diseased patients. Hence MIF levels for 80% of endometriosis cases were correctly identified. Consequently, this suggests that analysis for MIF levels can therefore be used to screen for endometriosis. Finding elevated levels of MIF in patients with endometriosis compared to MIF levels among the control group thus suggests that MIF can be a useful biomarker for endometriosis.

Certain presently preferred embodiments of my invention have been described herein; however, it will be apparent to those skilled in the art that variations and modifications of these embodiments shown and described may be made without departing from the spirit and scope of the invention. Accordingly, it is intended that the invention be limited only to the extent required by the appended claims and the applicable rules of law.

APPENDIX
Summary Table for Normal Male/Female Samples:

| | | | | | |
|---|---|---|---|---|---|
| | | Serum | EDTA | Heparin | Citrate |
| | | Adjusted | Adjusted | Adjusted | Adjusted |
| | | Conc. | Conc. | Conc. | Conc. |
| Sample ID | Age | (ng/ml) | (ng/ml) | (ng/ml) | (ng/ml) |

A. Male Samples from ZeptoMetrix received on Jun. 28, 2005

| | | | | | |
|---|---|---|---|---|---|
| 1 | 30 | 1.3 | 1.7 | 1.0 | 1.5 |
| 2 | 44 | 1.1 | 1.4 | 1.1 | 1.2 |
| 3 | 46 | 0.8 | 0.7 | 0.8 | 1.1 |
| 4 | 41 | 1.2 | 1.5 | 1.6 | 1.7 |
| 5 | 39 | 1.1 | 1.3 | 1.7 | 0.9 |
| 6 | 40 | 0.9 | 1.9 | 1.0 | 1.1 |
| 7 | 43 | 1.7 | 3.1 | 1.7 | 2.9 |
| 8 | 33 | 1.3 | 3.5 | 3.4 | 1.9 |
| 9 | 36 | 0.9 | 1.6 | 2.0 | 1.0 |
| 10 | 32 | 2.1 | 2.4 | 3.5 | 1.7 |
| 11 | 34 | 0.6 | 1.0 | 1.2 | 0.9 |
| 12 | 44 | 0.8 | 1.8 | 1.7 | 1.0 |
| 13 | 23 | 0.8 | 0.9 | 1.1 | 0.9 |
| 14 | 49 | 2.8 | 3.9 | 2.7 | 4.1 |
| 15 | 50 | 15.1 | 30.2 | 14.8 | 7.4 |
| 16 | 30 | 8.5 | 14.6 | 9.0 | 10.7 |
| 17 | 40 | 1.1 | 1.5 | 1.5 | 1.0 |
| 18 | 35 | 1.5 | 9.5 | 1.8 | 2.5 |
| 19 | 45 | 2.9 | 2.2 | 4.8 | 2.2 |
| 20 | 32 | 1.1 | 1.2 | 2.2 | 1.2 |
| 21 | 29 | 2.7 | 3.6 | 1.8 | 4.1 |
| 22 | 46 | 3.7 | 6.6 | 4.1 | 6.0 |
| 23 | 21 | 1.4 | 3.0 | 1.2 | 1.9 |
| 24 | 31 | 1.0 | 1.2 | 2.0 | Sample N/A |
| 25 | 45 | 10.9 | 31.8 | 31.7 | 32.2 |
| 26 | 52 | 15.1 | 20.1 | 15.4 | Sample N/A |
| 27 | 56 | 17.2 | 5.4 | 4.6 | 3.0 |
| Average | | 3.7 | 5.8 | 4.4 | 3.8 |
| (ng/ml) | | | | | |
| Median | | 1.3 | 2.2 | 1.8 | 1.7 |
| (ng/ml) | | | | | |

B. Female Samples from ZeptoMetrix received on Jun. 28, 2005

| | | | | | |
|---|---|---|---|---|---|
| 1 | 39 | 1.3 | 0.8 | 0.8 | 1.1 |
| 2 | 26 | 2.0 | 3.1 | 2.6 | 1.9 |
| 3 | 39 | 1.2 | 1.4 | Sample N/A | 2.8 |
| 4 | 44 | 1.7 | 1.4 | Sample N/A | 1.8 |
| 5 | 42 | 2.2 | 2.7 | Sample N/A | 2.3 |
| 6 | 44 | 0.9 | 1.4 | Sample N/A | 1.3 |
| 7 | 45 | 1.4 | 1.8 | Sample N/A | 1.6 |
| 8 | 47 | 1.2 | 5.6 | Sample N/A | 1.0 |
| 9 | 47 | 1.4 | 0.8 | Sample N/A | 1.4 |
| 10 | 61 | 1.2 | 1.5 | Sample N/A | 1.5 |
| 11 | 44 | 0.7 | 1.2 | 0.9 | 0.7 |
| 12 | 26 | 0.8 | 0.5 | 0.8 | 1.5 |
| 13 | 23 | 0.5 | 0.5 | 0.6 | 1.0 |
| 14 | 24 | 2.3 | 1.0 | 1.2 | 2.2 |
| 15 | 52 | 0.1 | 0.1 | 0.2 | 0.2 |
| 16 | 21 | 1.0 | 1.2 | 1.1 | 1.1 |
| 17 | 24 | 1.7 | 1.5 | 1.3 | 1.0 |
| 18 | 33 | 1.7 | 2.2 | 1.7 | 1.5 |
| 19 | 21 | 1.7 | 2.5 | 2.7 | 2.0 |
| 20 | 31 | 2.1 | 3.7 | 1.9 | 2.0 |
| 21 | 18 | 3.8 | 7.5 | 12.9 | 4.4 |
| 22 | 54 | 2.9 | 4.0 | 2.3 | 2.9 |
| 23 | 23 | 2.3 | 1.9 | 2.3 | 2.1 |
| 24 | 25 | 4.4 | 3.3 | 5.0 | 2.5 |
| 25 | 35 | 2.0 | 2.9 | 1.4 | 1.1 |
| 26 | 36 | 17.3 | 39.6 | 23.0 | 23.9 |
| 27 | 28 | 27.1 | 34.0 | 13.2 | 15.7 |
| 28 | 29 | 17.2 | 30.2 | 18.8 | 21.9 |
| 29 | 25 | 4.2 | 3.5 | 2.2 | 2.8 |
| 30 | 46 | 8.6 | 10.3 | 5.6 | 11.3 |
| 31 | 27 | 10.4 | 14.6 | 5.0 | 11.3 |
| 32 | 34 | 10.3 | 17.0 | 14.1 | 9.0 |
| 33 | 35 | 2.8 | 3.3 | 2.4 | Sample N/A |
| Average | | 4.3 | 6.3 | 5.0 | 4.3 |
| (ng/ml) | | | | | |
| Median | | 2.0 | 2.5 | 2.3 | 2.0 |
| (ng/ml) | | | | | |

I claim:

1. A method of diagnosing or confirming pancreatic cancer in a human that comprises determining serum macrophage migration inhibitory factor (MIF) levels using an Elisa immunoassay with a 1:10 dilution.

2. The method of claim 1 wherein a positive diagnosis for pancreatic cancer is made when the determined MIF level is above about 2.6 ng/ml.

3. The method of claim 1 which further comprises testing serum using MIF as a biomarker whereby a positive diagnosis for pancreatic cancer is made when the determined MIF level is above about 2.6 ng/ml.

4. A method of diagnosing pancreatic cancer in a human that comprises determining serum levels of MIF of above about 2.6 ng/ml using a modified ELISA immunoassay methodology.

5. A method of diagnosing endometriosis in a human that comprises determining serum levels of MIF using a modified ELISA immunoassay methodology.

6. The method of claim 5 where a diagnoses of endometriosis is made when the determined MIF level is greater than about 2.6 ng/ml.

7. A method of diagnosing or confirming pancreatic cancer in a human that comprises determining the serum levels of MIF using an immunoassay.

8. The method of claim 7 wherein said immunoassay is ELISA.

9. The method of claim 8 wherein said ELISA immunoassay uses a dilution of 1:10.

10. The method of claim 7 wherein a positive diagnosis of pancreatic cancer is made or confirmed when the MIF level is above about 2.6 ng/ml.
